(19) United States
(12) Patent Application Publication
(10) Pub. No.: US 2021/0134413 A1
(43) Pub. Date: May 6, 2021

(54) DIGITAL STORAGE BRACELET

(71) Applicant: SelfSafe, LLC, Katy, TX (US)

(72) Inventor: Edward A. Jacobson, Richmond, TX (US)

(21) Appl. No.: 17/086,004

(22) Filed: Oct. 30, 2020

Related U.S. Application Data
(60) Provisional application No. 62/928,848, filed on Oct. 31, 2019.

(51) Int. Cl. G16H 10/65; G06F 21/62; G06F 1/16

(52) U.S. Cl. CPC G16H 10/65; G06F 1/163; G06F 21/6245

(57) ABSTRACT

A storage bracelet includes an electronic storage assembly releasably engagable with an underside of a bracelet assembly that is securable about an appendage, wherein the electronic storage assembly is not removable from the bracelet assembly while the bracelet assembly is secured about the appendage. Methods to utilize the storage bracelet are also disclosed.

### BACKGROUND OF THE INVENTION

It is impossible to predict when one may require medical and other personal records. While such records may h available via internet or other network means, such networks are not always available when needed. Furthermore, records stored and accessible via the Internet are subject to being stolen or otherwise misused. It is beneficial for persons to have their records readily available in case of an emergency, or in the case of theft or some other miscarriage of events. In particular, it would be beneficial for travelers, both local and international, individuals with multiple residences, athletes and sports enthusiasts whom participate in various activities such as hiking, hiking, sailing and the like, as well as adults and/or children with life-threatening ailments or allergies to have their personal records readily available. In addition, such records may be beneficial for at risk populations such as the elderly, the disable, and to their caregivers should the need arise.

There is a need for a means to have personal records readily available, yet in a secure format to prevent theft.

### SUMMARY OF THE INVENTION

This summary is provided to introduce a selection of concepts that are further described below in the detailed description. This summary is not intended to identify key or essential features of the claimed subject matter, nor is it intended to be used as an aid in limiting the scope of the claimed subject matter.

Applicant has discovered an apparatus suitable to safely store a number of personal records in a secure format which is easily transported and readily available with readily available technology.

In one or more embodiments of the invention, a storage bracelet comprises an electronic storage assembly releasably engagable with an underside of a bracelet assembly that is securable about an appendage, wherein the electronic storage assembly is not removable from the bracelet assembly while the bracelet assembly is secured about the appendage.

In one or more embodiments of the invention, a method comprises the steps of providing a storage bracelet according to one or more of the embodiments disclosed herein; disengaging the electronic storage assembly from the bracelet assembly; electronically storing a personal record on the electronic storage assembly; engaging the electronic storage assembly with the bracelet assembly, and securing the storage bracelet about an appendage.

### BRIEF DESCRIPTION OF THE DRAWINGS

Embodiments according to the instant application are described with reference to the following figures. The same numbers are used throughout the figures to reference like features and components.

FIG. 1 is a top perspective view of a digital storage bracelet according to embodiments of this disclosure;

FIG. 2 is a side sectional view of the assembled digital storage bracelet of FIG. 1;

FIG. 3 is a side sectional view of the digital storage bracelet of FIG. 2 with the resilient body of the bracelet assembly bent up in preparation for removal of the electronic storage assembly;

FIG. 4 is a top perspective view of a digital storage bracelet shown in FIG. 1, showing the bracelet assembly with the electronic storage assembly removed from the underside of the bracelet assembly according to embodiments of this disclosure;

FIG. 5 is a bottom perspective view, showing the underside of the bracelet assembly shown in FIG. 4 with the electronic storage assembly removed according to embodiments of this disclosure;

FIG. 6 is an expanded perspective view of a two-part digital storage assembly according to embodiments of this disclosure;

FIG. 7 is a perspective view of the assembled two-part digital storage assembly shown in FIG. 4, according to embodiments of this disclosure;

FIG. 8 shows a perspective view of a digital storage bracelet in a closed position, according to embodiments of this disclosure; and

FIG. 9 shows a digital storage bracelet disposed about a human appendage according to embodiments of the disclosure.

### DETAILED DESCRIPTION OF THE INVENTION

At the outset, it should be noted that in the development of any such actual embodiment, numerous implementation-specific decisions must be made to achieve the developer's specific goals, such as compliance with system related and business related constraints, which will vary from one implementation to another. Moreover, it will be appreciated that such a development effort might be complex and time consuming but would nevertheless be a routine undertaking for those of ordinary skill in the art having the benefit of this disclosure.

It is to be understood that various embodiments of the present disclosure will be described with reference to the accompanying drawings. Accordingly, those of ordinary skill in the art will recognize that modifications, equivalents, and/or alternatives of the various embodiments described herein can be made without departing from the scope and spirit of the present disclosure. With regard to description of drawings, similar components may be marked by similar reference numerals.

For use herein, the expressions “have”, “may have”, “include”, “comprise”, “may include”, and “may comprise” indicate the existence of corresponding features (e.g., such as numeric values, functions, operations, or components) but do not exclude the presence of additional features.

In the present disclosure, expressions such as, “A or B”, “at least one of A and/or B”, and “one or more of A and/or B”, may include any and all combinations of one or more of the associated listed items. For example, the terms “A or B”, and “at least one of A or B” may refer to the case (1) where A is included, (2) where B is included, or (3) where both A and B are included.

Terms such as “first”, “second”, and the like used herein may refer to various elements of various embodiments of the present disclosure, but do not limit the elements. For example, such terms are used only to distinguish an element from another element and do not limit the order and/or priority of the elements. For example, a first user device and a second user device may represent different user devices irrespective of sequence or importance. For example, without departing from the scope of the present disclosure, a first element may be referred to as a second element, and similarly, a second element may be referred to as a first element.

It will be understood that when an element (for example, a first element) is referred to as being operatively or communicatively “coupled with/to” or “connected to” another element (for example, a second element), it can be directly coupled with/to or connected to the other element or an intervening element (for example, a third element) may be present.

As used herein, the expression “configured to” used herein may be used interchangeably with, for example, the expression “suitable for”, “having the capacity to”, “designed to”, “adapted to”, “made to”, or “capable of”. The term “configured to (or set to)” does not mean only “specifically designed to” in hardware. Instead, the expression “a device configured to” may mean that the device is “capable of” operating together with another device or other components. For example, an “electronic memory assembly” configured to (or set to) store data for subsequent retrieval, refers to any such memory module or modules, with the associated circuitry, power source, and programming which render it capable of performing the corresponding operation of storage and retrieval utilizing a generic-purpose processor (e.g., a central processing unit (CPU) or an application processor) which may perform corresponding operations by executing one or more software programs which are stored on the memory device.

The terms used herein are used to describe specific embodiments of the present disclosure and are not intended to limit the scope of the present disclosure. The terms of a singular form may include plural forms unless otherwise specified. Unless otherwise defined herein, all the terms used herein, which include technical or scientific terms, may have the same meaning as that generally understood by a person skilled in the art to which the present disclosure pertains. It will be further understood that terms, which are defined in a dictionary and commonly used, should also be interpreted as is customary in the relevant related art and not in an idealized or overly formal manner unless expressly so defined herein. In some cases, even if terms are defined in the specification, they may not be interpreted to exclude embodiments of the present disclosure.

In various embodiments of the present disclosure, the electronic components referred to in embodiments of the device are not limited to currently known devices, but may include new electronic devices suitable for the intended purpose which are subsequently produced due to the development of new technologies.

The term “user” of a device refers to a person who uses an electronic device in any capacity. Accordingly, for purposes herein, the user may be the wearer of the storage bracelet, and/or a health care provider or other individual who retrieves records from the storage bracelet.

In embodiments, a storage bracelet, also referred to herein as an electronic storage bracelet or a digital storage bracelet, comprises an electronic storage assembly releasably engageable with an underside of a bracelet assembly that is securable about an appendage, wherein the electronic storage assembly is not removable from the bracelet assembly while the bracelet assembly is secured about the appendage.

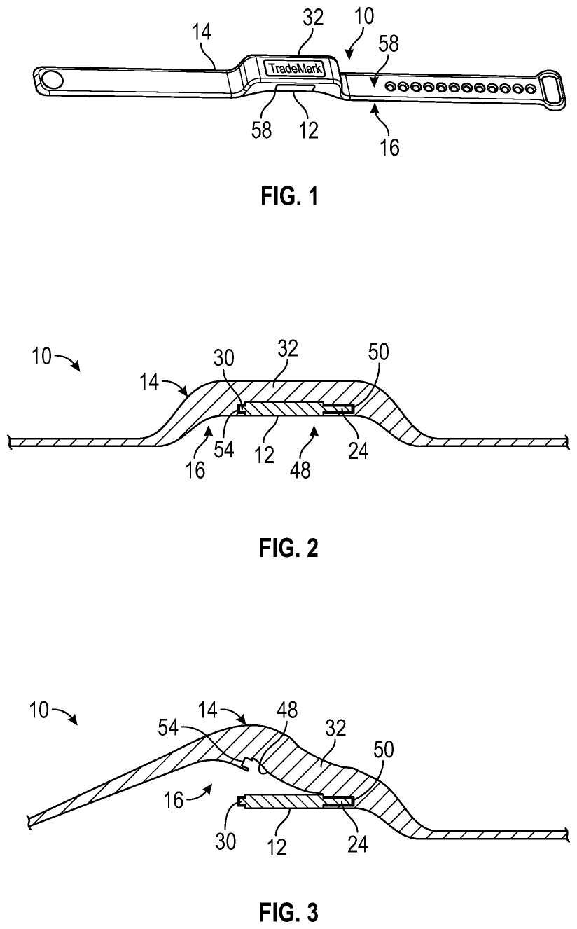

Turning to FIGS. 1 and 2, in one or more embodiments, a storage bracelet, generally referred to as 10, comprises an electronic storage assembly 12 releasably engageable with an underside 16 of a bracelet assembly 14 that is securable about an appendage 18 (see FIG. 7), which may be an arm/wrist or a leg/ankle, for example. In one or more embodiments, the electronic storage assembly 12 is not removable from the bracelet assembly 14 while the storage bracelet 10 is secured about the appendage 18.

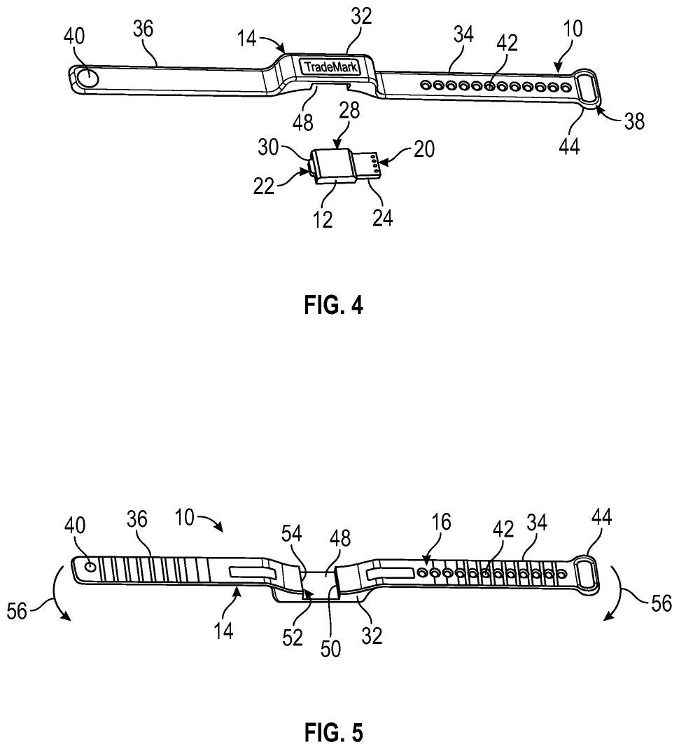

However, as best seen in FIG. 3, the bracelet assembly 14 comprises a resilient body 32, which can be bent upwardly when the bracelet 10 is disengaged from the appendage 18 to release a locking tab 30 on the electronic storage assembly 12 from a slot 54 formed in a second end 52 of the resilient body 32, in preparation for releasing the electronic connector 24 (see FIG. 4) of the electronic storage assembly 12 from the first end 50 of the resilient body 32.

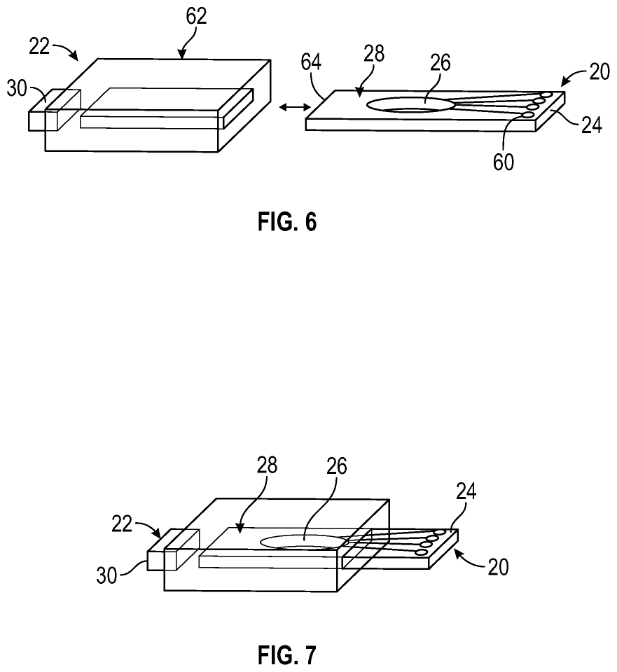

As shown in FIG. 4, in one or more embodiments, the electronic storage assembly 12 comprises a first end 20 separated from second end 22. The first end 20 comprises an electronic connector 24 of an electronic storage device, generally indicated as 28, in electrical communication with an electronic memory assembly 26 (see FIGS. 6 and 7). Suitable electronic storage devices 28 include so-called USB thumb drives, and other solid-state electronic storage devices. In embodiments, the second end 22 comprises a locking tab 30 extending away from the first end 20.

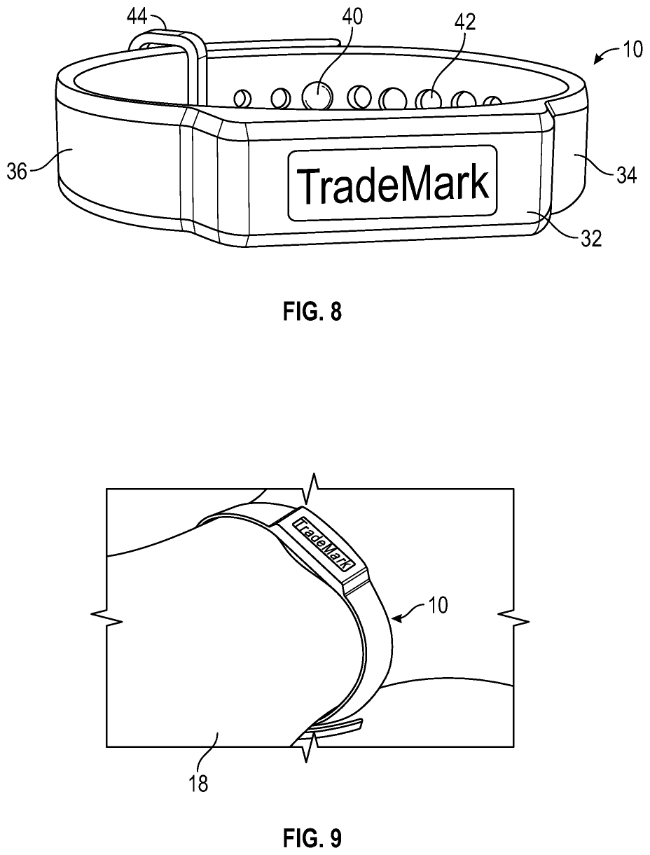

In embodiments, the bracelet assembly 14 comprises an elongated, resilient body 32 having straps 34 and 36 extending away from either end. The straps 34 and 36 terminate in a cooperative closure assembly, generally indicated as 38, for securing the storage bracelet 10 about the appendage 18. As shown in FIG. 2, in embodiments the cooperative closure assembly 38 comprises a loop 44 and a plurality of holes 42 disposed through strap 34, and an engagement pin 40 disposed through strap 36 which engages one of the plurality of holes 42, as shown in FIG. 8.

In embodiments, the bracelet assembly 14 further comprises a compartment 48 formed into the underside 16 of the resilient body 32 having a first end 50 dimensioned and arranged to receive and releasably engage at least a portion of the first end 20 of the electronic storage assembly 12, and a second end 52 comprising a slot 54 arranged to releasably engage the locking tab 30 of the second end 22 of the electronic storage assembly 12. In embodiments, the resilient body 32 comprises resilience as indicated by arrows 56 to bend toward an obverse side 58 as shown in FIG. 3, to separate the first end 50 and the second end 52 of the compartment 48 for insertion and removal of the electronic storage assembly 12 from the bracelet assembly 14.

In embodiments, the engagement of first end 50 of the compartment 48, with at least a portion of the first end 20 of the electronic storage assembly 12 is via a tab and slot arrangement with at least a portion of the electronic connector 24 acting as the tab and disposed within a hollow chamber disposed into the first end 50 of the compartment 48 acting as the slot. In one or more embodiments, there exists a water-resistant seal between the bracelet assembly 14 and the portion of the first end 20 of the electronic storage assembly 12 disposed within the hollow chamber of the body 34.

Returning to FIG. 1, in one or more embodiments, the electronic storage assembly 12, when engaged with the bracelet assembly 14, is flush with and/or forms a portion of a profile 58 of the body 32. Accordingly, with the bracelet engaged about an appendage, it is unable to bend toward the obverse side 58 to separate the first end 50 and the second end 52 of the compartment 48 to allow removal of the electronic storage assembly 12 from the bracelet assembly 14.

In one or more embodiments, the electronic storage device 28 comprises a USB storage device, a lightning storage device, a thunderbolt storage device, or a combination thereof. Suitable USB storage devices include those with USB-A connectors, USB C connectors, USB-mini connectors, and USB-micro connectors. Accordingly, in one or more embodiments, the electronic connector 24 comprises a USB-A format, a USB-mini format, a USB-micro format, a USB-C format, or a lightning connector format.

As shown in FIG. 4, in one of more embodiments, first end 20 of the electronic storage assembly 12 is at least partially formed by a male portion (e.g., the electronic connector 24) of the electronic storage device 28, and may further comprise the electronic connections 60 suitable for insertion into a corresponding female port of a computer as is commonly known in the art.

As shown in FIG. 6, in one or more embodiments, the electronic storage assembly 12 comprises two or more components. In an embodiment, the second end 22 of the electronic storage assembly 12 is formed from or serves as a protective cover 62 releasably and sealing engagable about the end 64 of the electronic storage device 28 located opposite the end comprising the electronic connector 24. In such an embodiment, the protective cover 62 further comprises the locking tab 30 extending away therefrom.

In one or more embodiments, the body 32 and at least a portion of the electronic storage assembly 12 form a water resistant enclosure in which the electronic storage device 28 is at least partially located, preferably having an ingress protection classification about the electronic connector of IP63, preferably IP67, more preferably IP68, when determined according to IEC standard 60529, or an equivalent thereof.

In one or more embodiments, the storage bracelet 10 consists essentially of the electronic storage assembly 12 and the bracelet assembly 14. In other words, the storage bracelet 10 does not include any additional sensors, batteries, monitors, and/or the like, but is instead simply a passive information storage device which must be engaged with a corresponding computer to retrieve any information stored in the electronic storage assembly 12.

As shown in FIG. 8, in one or more embodiments, the first strap 34 comprises a plurality of sizing holes 42 disposed therethrough, and +terminates in an opening 44 through which an end of the second strap 36 is disposable through, and the end of the second strap comprises an engagement pin 40 preferably having a bulbus head, sized to be disposable through, and to frictionally engage with one or more of the sizing holes 42 disposed through the first strap 34.

In one or more embodiments, the bracelet assembly 14 and optionally at least a portion of the electronic storage assembly 12 comprises flexible elastomeric material, having a Shore A hardness from about 10 to less than or equal to about 75, preferably from about 45 to about 60, when determined according to ASTM D2240 or an equivalent thereof. In one or more embodiments, the elastomer comprises or consists essentially of a polysiloxane, i.e., a silicone rubber.

In one or more embodiments, the storage bracelet 10 further comprises one or more records stored on the electronic storage assembly 12 in computer readable format. In embodiments, the record is stored in an encrypted format and password protected from removal and/or editing.

In embodiments of the invention, a method comprises providing a storage bracelet according to any one or more embodiments disclosed herein, disengaging the electronic storage assembly from the bracelet assembly; electronically storing a personal record on the electronic storage assembly; engaging the electronic storage assembly with the bracelet assembly; and securing the storage bracelet about an appendage.

In embodiments, this method further includes removing the storage bracelet from the appendage, disengaging the electronic storage assembly from the bracelet assembly, and electronically retrieving the personal record stored on the electronic storage assembly, e.g., via inserting the electronic connector into an appropriate port on a computer and retrieving the information stored thereon. In embodiments, the retrieval of the information requires entering of a predetermined password or other security information.

Having described the various aspects of the compositions herein, further specific embodiments of the invention include those set forth in the following paragraphs:
A1. A storage bracelet comprising:
   an electronic storage assembly releasably engagable with an underside of a bracelet assembly that is securable about an appendage, wherein the electronic storage assembly is not removable from the bracelet assembly while the bracelet assembly is secured about the appendage.
A2. The storage bracelet according to embodiment A1, wherein the electronic storage assembly comprises a first end separated from second end, the first end comprising an electronic connector of an electronic storage device in electrical communication with an electronic memory assembly;
   the second end comprising a locking tab extending away therefrom;
   the bracelet assembly comprising an elongated, resilient body having straps extending away from either end which terminate in a cooperative closure assembly for securing the bracelet about the appendage;
   a compartment formed into the underside of the resilient body having a first end dimensioned and arranged to receive and releasably engage at least a portion of the first end of the electronic storage assembly, and a second end comprising a slot arranged to releasably engage the locking tab of the second end of the electronic storage assembly;
   the resilient body comprising resilience to bend toward an obverse side to separate the first and second ends of the compartment for insertion and removal of the electronic storage assembly from the bracelet assembly.
A3. The storage bracelet according to embodiment A1 or A2, wherein the engagement of the first end of the compartment with at least a portion of the first end of the electronic storage assembly forms a water-resistant seal there between.
A4. The storage bracelet according to any one of embodiments A1 through A3, wherein the electronic storage assembly, when engaged with the bracelet assembly, is flush with a profile of the body.
A5. The storage bracelet according to any one of embodiments A1 through A4, wherein the electronic storage device comprises a USB storage device, a lightning storage device, a thunderbolt storage device, or a combination thereof.
A6. The storage bracelet according to any one of embodiments A1 through A5, wherein a first strap comprises a plurality of sizing holes disposed therethrough, and terminates in an opening through which an end of the second strap is disposable through, and wherein the end of the second strap comprises an engagement pin having a bulbus head, sized to be disposable through, and to frictionally engage with one or more of the sizing holes disposed through the first strap.
A7. The storage bracelet according to any one of embodiments A1 through A6, wherein the first end of the electronic storage assembly is at least partially formed by a male portion of the electronic storage device comprising the electronic connector, suitable for insertion into a corresponding female port of a computer.
A8. The storage bracelet according to embodiment A7, wherein the second end of the electronic storage assembly is formed from a protective cover releasably and sealing engagable about an end of the electronic storage device located opposite the electronic connector, and further comprising the locking tab extending away therefrom.
A9. The storage bracelet according to embodiment A8, wherein the body and a portion of the electronic storage assembly form a water resistant enclosure in which the electronic storage device is at least partially located, having an ingress protection classification about the electronic connector of IP63, preferably IP67, more preferably IP68, when determined according to IEC standard 60529, or an equivalent thereof.
A10. The storage bracelet according to any one of embodiments A1 through A9, wherein the electronic connector comprises a USB-A format, a USB-mini format, a USB-micro format, a USB-C format, or a lightning connector format.
A11. The storage bracelet according to any one of embodiments A1 through A10, consisting essentially of the electronic storage assembly and the bracelet assembly.
A12. The storage bracelet according to any one of embodiments A1 through A11, comprising a rubber having a Shore A hardness from about 10 to less than or equal to about 75, preferably from about 45 to about 60, when determined according to ASTM D2240 or an equivalent thereof.
A13. The storage bracelet according to embodiment A12, wherein the rubber comprises a polysiloxane.
A14. The storage bracelet according to any one of embodiments A1 through A13, further comprising one or more records stored on the electronic storage assembly in computer readable format.
A15. The storage bracelet according to embodiment A14, wherein the record is stored in an encrypted format and password protected from removal and/or editing.
A16. A method comprising:
   providing a storage bracelet according to any one of embodiments A1 through A15;
   disengaging the electronic storage assembly from the bracelet assembly;
   electronically storing a personal record on the electronic storage assembly;
   engaging the electronic storage assembly with the bracelet assembly; and
   securing the storage bracelet about an appendage.
A17. The method according to embodiment A16, further comprising removing the storage bracelet from the appendage; disengaging the electronic storage assembly from the bracelet assembly; and electronically retrieving the personal record stored on the electronic storage assembly.
A18. The method according to embodiment A17, further comprising entering a password prior to retrieving the personal record.

Certain embodiments and features have been described using a set of numerical upper limits and a set of numerical lower limits. It should be appreciated that ranges from any lower limit to any upper limit are contemplated unless otherwise indicated. Certain lower limits, upper limits and ranges appear in one or more claims below. All numerical values are “about” or “approximately” the indicated value, and take into account experimental error and variations

As used herein, the phrases “substantially no,” and “substantially free of ” are intended to mean that the subject item is not intentionally used or added in any amount, but may be present in very small amounts existing as impurities resulting from environmental or process conditions.

To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

I claim:

1. A storage bracelet comprising:
an electronic storage assembly releasably engagable with an underside of a bracelet assembly that is adapted to be secured about an appendage, wherein the electronic storage assembly is not removable from the bracelet assembly while the bracelet assembly is secured about the appendage.

2. The storage bracelet of claim 1, wherein the electronic storage assembly comprises a first end spaced from a second end, the first end comprising an electronic connector of an electronic storage device in electrical communication with an electronic memory assembly;
the second end comprising a locking tab extending away therefrom;
the bracelet assembly comprising an elongated, resilient body having straps extending away from either end which terminate in a cooperative closure assembly for securing the bracelet about the appendage;
a compartment formed into the underside of the resilient body having a first end dimensioned and arranged to receive and releasably engage at least a portion of the first end of the electronic storage assembly, and a second end comprising a slot arranged to releasably engage the locking tab of the second end of the electronic storage assembly;
the resilient body comprising resilience to bend toward an obverse side to separate the first and second ends of the compartment for insertion and removal of the electronic storage assembly from the bracelet assembly.

3. The storage bracelet of claim 2, wherein the engagement of the first end of the compartment with at least a portion of the first end of the electronic storage assembly forms a water-resistant seal therebetween.

4. The storage bracelet of claim 2, wherein the electronic storage assembly, when engaged with the bracelet assembly, is flush with a profile of the resilient body.

5. The storage bracelet of claim 2, wherein the electronic storage device comprises a USB storage device, a lightning storage device, a thunderbolt storage device, or a combination thereof

6. The storage bracelet of claim 2, wherein a first strap comprises a plurality of sizing holes disposed therethrough, and terminates in an opening through which an end of the second strap is disposable through, and wherein the end of the second strap comprises an engagement pin having a bulbus head, sized to be disposable through, and to frictionally engage with one or more of the sizing holes disposed through the first strap.

7. The storage bracelet of claim 2, wherein the first end of the electronic storage assembly is at least partially formed by a male portion of the electronic storage device comprising the electronic connector, suitable for insertion into a corresponding female port of a computer.

8. The storage bracelet of claim 7, wherein the second end of the electronic storage assembly is formed from a protective cover releasably and sealing engagable about an end of the electronic storage device located opposite the electronic connector, and further comprising the locking tab extending away therefrom.

9. The storage bracelet of claim 8, wherein the body and a portion of the electronic storage assembly form a water resistant enclosure in which the electronic storage device is at least partially located, having an ingress protection classification about the electronic connector of IP63, preferably IP67, more preferably IP68, when determined according to IEC standard 60529, or an equivalent thereof.

10. The storage bracelet of claim 1, wherein the electronic connector comprises a USB-A format, a USB-mini format, a USB-micro format, a USB-C format, or a lightning connector format.

11. The storage bracelet of claim 1, consisting essentially of the electronic storage assembly and the bracelet assembly.

12. The storage bracelet of claim 1, comprising a rubber having a Shore A hardness from about 10 to less than or equal to about 75, preferably from about 45 to about 60, when determined according to ASTM D2240 or an equivalent thereof.

13. The storage bracelet of claim 12, wherein the rubber comprises a polysiloxane.

14. The storage bracelet of claim 1, further comprising one or more records stored on the electronic storage assembly in computer readable format.

15. The storage bracelet of claim 14, wherein the record is stored in an encrypted format and password protected from removal and/or editing.

16. A method comprising:
providing a storage bracelet according to claim 1;
disengaging the electronic storage assembly from the bracelet assembly;
electronically storing a personal record on the electronic storage assembly;
engaging the electronic storage assembly with the bracelet assembly; and
securing the storage bracelet about an appendage.

17. The method of claim 16, further comprising removing the storage bracelet from the appendage; disengaging the electronic storage assembly from the bracelet assembly;
and electronically retrieving the personal record stored on the electronic storage assembly.

18. The method of claim 17, wherein the personal record is password-protected and further comprising entering the password prior to retrieving the personal record.
